(19) United States
(12) Patent Application Publication
(10) Pub. No.: US 2004/0047838 A1
(43) Pub. Date: Mar. 11, 2004

(54) Tissue flap angiogenesis

(75) Inventors: Ronald G. Crystal, New York, NY (US); Todd K. Rosengart, Highland Park, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Cornell Research Foundation, Inc., Ithaca, NY (US)

(21) Appl. No.: 10/657,879

(22) Filed: Sep. 9, 2003

Related U.S. Application Data
(63) Continuation of application No. 09/406,345, filed on Sep. 28, 1999, now abandoned.

(51) Int. Cl. A61K 48/00; C12N 15/861

(52) U.S. Cl. 424/93.2; 435/456

(57) ABSTRACT

The present invention provides a method of increasing vascularity in a tissue flap. The method comprises contacting a tissue flap with a viral vector, which viral vector comprises a nucleic acid sequence encoding an angiogenic factor, whereby the nucleic acid sequence encoding the angiogenic factor is expressed in the tissue flap and vascularity in the tissue flap is increased.

### CROSS-REFERENCE TO RELATED PATENT APPLICATIONS

This patent application is a continuation of copending U.S. patent application Ser. No. 09/406,345, filed Sep. 28, 1999.

### TECHNICAL FIELD OF THE INVENTION

The invention relates to the field of angiogenesis. In particular, the invention relates to methods for promoting angiogenesis and reducing the rate of necrosis in tissue flaps, for example during tissue flap surgery.

### BACKGROUND OF THE INVENTION

Tissue flaps (also referred to herein as flaps) are used in (and produced during) many types of surgical procedures, particularly reconstructive surgery in a variety of indications to correct a multitude of tissue defects. For example, flaps may be used to resurface (or can be created by incision in) a variety of wounds about the head, neck, extremities and trunk or they may be employed to cover exposed tendons, bones or major blood vessels. Flaps may be used about the face where color match and contour are important or they may be used to close wounds having a poor blood supply as where wound circulation would not support a skin graft. A tissue flap traditionally refers to skin and subcutaneous tissue (or muscle, bone or other tissue) along with the entire vascular plexuses, thereby bringing a large supply of tissue and an intact blood supply to the site of injury. Modem surgical techniques have expanded the traditional definition of a tissue flap to encompass free, microvascular flaps that may be anastomosed to an existing blood supply at or near the site of injury.

Tissue flaps are also produced during surgery. For example, tissue flaps are produced during breast reconstruction surgery wherein skin, fat and the rectus muscle from the abdomen are removed and re-located to the chest to make the new breast. Similarly, tissue flaps can be produced temporarily during surgical procedures wherein surgical incisions are made in a patient.

A persistent problem in the use of tissue flaps has been that of survival of the flap due to a diminished blood supply, which is a leading reason for failure of the flap and consequent unsatisfactory management of a wound. Various factors which influence the failure of these tissue flaps include extrinsic factors such as compression or tension on the flap, kinking of the pedicle, infection, hematoma, vascular disease, hypotension and abnormal nutritional states. Ischemia has also been postulated as playing a role in skin flap failure although the precise etiology has not been conclusively elucidated. For example, Reinisch (Plastic and Reconstructive Surgery, 54, 585-598 (1984)) theorizes that the ischemia is due to the opening of A-V shunts with resultant non-nutritive blood flow to the effected area. On the other hand, Kerrigan (Plastic and Reconstructive Surgery, 72, 766-774 (1983)) speculates that the ischemia is due to arterial insufficiency causing insignificant blood flow in the distal portion of the flap.

Because failure of these flaps can have deleterious consequences for the patient, various measures have been taken in the past to attempt to salvage failing flaps. Such measures include re-positioning the flap, topical cooling of the region, hyperbaric oxygen, as well as the administration of various drugs. Among the drugs that have been used are dimethyl sulfoxide, histamine, isoxuprine and prostaglandin inhibitors. Additionally, various sympatholytic agents such as reserpine, phenoxybenzamine, propranolol guanethidine and 6-hydroxy-dopa have been used, as well as rheologic-altering agents such as dextran, heparin and pentoxifylline. Systemic steroids have been used in an attempt to increase body tolerance to ischemia, as has topical applications of flamazine.

U.S. Pat. No. 4,599,340 (Silver et al.) teaches a method of reducing tissue flap necrosis in a patient undergoing reconstructive surgery by administering an affective amount of a channel blocking drug. Such drugs are capable of lowering blood pressure and have a wide range of applicability in treatment of injury and disease. However, studies in recent years have generated concerns that calcium channel blocking drugs can be dangerous for some individuals. Moreover, the use of such drugs has been associated with undesirable side effects.

Thus, none of the above-referenced treatment modalities or drugs used in prior attempts to reduce tissue flap necrosis have been entirely satisfactory or met with widespread acceptance in the medical community. Hence a need still exists for a means of reducing tissue flap necrosis (and the resultant failure of the flap) for use in reconstructive surgery.

Angiogenesis, i.e. the growth of new capillary blood vessels, is a process that is crucial to the proper healing of many types of wounds. Consequently, factors that are capable of promoting angiogenesis are useful as wound healing agents. Such factors include fibroblast growth factor (FGF) and vascular endothelial growth factor (VEGF). Angiogenesis is a multi-step process involving capillary endothelial cell proliferation, migration and tissue penetration.

Recent research has shown that application of an angiogenic protein (e.g., FGF) can promote flap survival in rats. See Rashid et al., Plast. Reconstr. Surg., 103, 941-48 (1999); Bayati et al., Plast Reconstr. Surg., 101, 1290-95 (1998). Researchers have shown similar results for direct injection of VEGF. See Kryger et al., Ann. Plast. Surg., 43, 172-78 (1999); Wei, Chung Kuo Hsiu Fu Chung Chien Wai Ko Tsa Chih, 11, 376-78 (1997); Padubidri et al., Ann Plast. Surg., 37, 604-11 (1996).

Delivery of an angiogenic protein to a wound to promote angiogenesis and wound healing has been accomplished by a variety of methods including direct application to the site of the wound, soaking the skin or flap that is being treated, intravenous injection, and by a using micrometering pump as a parenteral solution. The disadvantages of such techniques include the need for repeated treatments in order to sustain a therapeutic result. Moreover, it is often not practical and/or economical to obtain the necessary and/or commercial quantities of the angiogenic protein for such treatments.

Recently, Taub et al., J. Reconstr. Microsurg. 14, 387-90 (1998), infused rat abdominal skin flaps with a VEGF gene with apparently mixed results in the survivability of such flaps after treatment. In another reference, Taub et al., Plast Reconstr. Surg., 102, 2033-39 (1998), discloses delivery of a cDNA encoding VEGF in connection with a liposome-mediated gene transfer system with apparently better results over a short time period.

In view of the uncertainty and problems associated with such techniques, as well as the less than satisfactory results of other techniques, there remains a need for alternative methods of promoting angiogenesis in tissue flaps. The present invention provides a method of promoting angiogenesis and preventing necrosis in tissue flaps. In particular, the present invention provides for the administration of an angiogenic factor to a tissue flap so as to promote angiogenesis in the tissue flap. These and other advantages of the present invention will become apparent from the description of the present invention herein.

### BRIEF SUMMARY OF THE INVENTION

The present invention provides a method of increasing vascularity in a tissue flap. The method comprises contacting a tissue flap with a viral vector, which viral vector comprises a nucleic acid sequence encoding an angiogenic factor, whereby the nucleic acid sequence encoding the angiogenic factor is expressed in the tissue flap and vascularity in the tissue flap is increased.

### DETAILED DESCRIPTION OF THE INVENTION

The present invention provides a method of increasing vascularity of a tissue flap. More particularly, the method comprises contacting a tissue flap with a viral vector that comprises a nucleic acid sequence encoding an angiogenic factor. The nucleic acid sequence encoding the angiogenic factor is expressed in the tissue flap, and vascularity in the tissue flap is thereby increased.

Delivery of a nucleic acid sequence encoding an angiogenic factor using a viral vector-mediated approach is advantageous since it provides high concentrations of the angiogenic factor for a sustained period. Such sustained delivery is quite useful inasmuch as many angiogenic factors, such as VEGF, have a very short biologic half-life (e.g., 6 minutes for VEGF) (Takeshita et al., J. Clin. Invest., 93, 662-670 (1994)).

The viral vector of the present invention serves to transfer coding information to a host cell which is (at least in part) of viral origin. Any suitable viral vector can be used in the context of the present invention. Preferably, an adenoviral vector is utilized in the present inventive method. Thus, an adenoviral vector utilized in accordance with the present invention can encompass any adenoviral vector that is appropriate for the introduction of nucleic acids into eukaryotic cells and is capable of functioning as a vector as that term is understood by those of ordinary skill in the art. An adenoviral vector in the context of the present invention contains one or more nucleic acid sequences that encode and are expressed to produce an angiogenic factor. Such sequences may also encode other therapeutic proteins or therapeutic mRNA, possibly one or more enhancers or silencers, promoters, and the like.

Adenovirus vectors used in the context of the present invention can be (or be based upon adenovirus selected from) any serotype of adenovirus (see, e.g., Fields Virology, Fields et al. (eds.), 3rd Ed., NY: Raven Press, 1996, pp. 2111-2171). Preferably, the adenoviral vector is of (or produced from) a serotype that can transduce and/or infect a human cell. Desirably, the adenovirus comprises a complete adenoviral virus particle (i.e., a virion) consisting of a core of nucleic acid and a protein capsid, or comprises a protein capsid to which DNA comprising a therapeutic gene is appended, or comprises a naked adenoviral genome, or is any other manifestation of adenovirus as described in the art and which can be used to transfer a therapeutic gene. In the context of the present invention, any suitable adenoviral genome can serve as, or be a part of, the adenoviral vector. Preferred adenoviral genomes include those derived from Ad5 and Ad2, which are easily isolated from infected cells, are commercially available, or are generally available from those skilled in the art who routinely maintain these viral stocks.

For the purpose of this invention, the adenoviral vector employed for transfer of the angiogenic factor can be wild-type (i.e., replication-competent). However, it is not necessary that the genome of the employed adenovirus be intact. In fact, to prevent the virus from usurping host cell functions and ultimately destroying the cell, the adenovirus can be inactivated prior to its use, for instance, by UV irradiation. Alternately, the adenovirus can comprise genetic material with at least one modification therein, which can render the virus replication-deficient. For example, an adenoviral vector can be deleted in the E1 region, or the E1 and E3 regions, of the adenoviral genome. Alternatively, the adenoviral vector can be a “multiply deficient” adenoviral vector having deletions in two or more regions essential for viral replication, for example, the E1 and E4 regions, in addition to optionally the non-essential E3 region. Such vectors are more completely described in WO 95/34671.

Thus, the adenovirus can consist of a gene encoding an angiogenic factor linked to an adenoviral capsid, and thus may not possess an adenoviral genome. Moreover, the virus can be coupled to a DNA-polylysine complex containing a ligand (e.g., transferrin) for mammalian cells such as has been described in the art.

Modifications to the adenoviral genome in an adenoviral vector suitable for use in the present invention can include, but are not limited to, addition of a DNA segment, rearrangement of a DNA segment, deletion of a DNA segment, replacement of a DNA segment, methylation of unmethylated DNA, demethylation of methylated DNA, and introduction of a DNA lesion. For the purpose of this invention, a DNA segment can be as small as one nucleotide and as large as 36 kilobase pairs (kb) (i.e., the size of the adenoviral genome) or, alternately, can equal the maximum amount which can be packaged into an adenoviral virion (i.e., about 38 kb).

Such modifications to the adenoviral genome can render the adenovirus replication-deficient. Preferably, however, the modification does not alter the ability of the adenovirus to bind to a suitable cell surface receptor. Preferred modifications to the adenoviral genome include modifications in the E1, E2, E3, and/or E4 regions. The vector according to the invention also can comprise a ligation of adenovirus sequences with other vector sequences.

Adenoviral vectors have the aforementioned properties that make them ideal for the delivery of a nucleic acid sequence encoding an angiogenic factor to a tissue flap as described herein. For instance, adenoviral vectors are effective at transferring genes to tissues with high levels of expression of the gene for at least one week. This is particularly advantageous in view of the short half-life of many angiogenic factors. Moreover, the self-limited nature of adenoviral-mediated gene expression means a decreased (and decreasing over time) risk of evoking too much angiogenesis in the target tissue. The nucleic acid sequence transferred by an adenoviral vector functions in an epichromosomal position, in contrast to adeno-associated virus and retrovirus vectors that integrate the exogenous gene into the chromosome of the target cell, and thus carry the risk of inappropriately delivering the angiogenic stimulus long after it is needed, and the risk of interference with the regulation/expression of an endogenous gene. Furthermore, adenovirus vectors achieve gene transfer to both dividing and non-dividing cells with high levels of efficiency, and produce localized and sustained levels of protein expression in a variety of tissue, such as adipose, muscle, and vascular endothelium.

The angiogenic factor of the present invention can be any suitable angiogenic factor. Preferably, the angiogenic factor comprises or is an angiogenic protein or peptide sequence. Nucleic acid sequences encoding the following angiogenic growth factors, and which have been described in the art, can be used according to the present invention: vascular endothelial cell growth factor (VEGF also known as VPF), acidic fibroblast growth factor (aFGF), basic fibroblast growth factor (bFGF), transforming growth factor, alpha and beta tumor necrosis factor, platelet-derived growth factor, and angiogenin. More preferably, the angiogenic factor comprises a growth factor such as FGF or VEGF. Even more preferably the angiogenic factor is a vascular endothelial growth factor (VEGF).

The vascular endothelial growth factor (VEGF) used in the present invention can be any suitable VEGF, including naturally occurring VEGF, a modified VEGF and/or angiogenic fragments thereof. For example the VEGF can be selected from the group comprising VEGF121, VEGF145, VEGF165, and VEGF189. Preferably, the VEGF is VEGF121.

The present invention can be utilized with respect to any suitable tissue flap, e.g., tissue flaps produced during surgical procedures, as well as tissue flaps used to treat wounds. The tissue flaps can be completely disassociated flaps of tissue suitable for reconnection or application, or sections of tissue which are substantially cut away from, but remain connected to, an animal host, for example a tissue flap generated during surgical incision. The tissue flap can be composed of suitable tissue such as skin, subcutaneous tissue, muscle, bone, vascular plexuses tissue, microvascular flaps, and combinations thereof.

The present invention can be used in a wide range of tissues that compose surgical flaps. For example, the present invention can be useful in promoting angiogenesis and reducing the rate of necrosis in tissue flaps used in, or generated by, a wide range of surgical techniques. Many procedures using or generating such tissue flaps are well known in the art and include the transverse rectus abdominus myocutaneous flap procedure (or TRAM procedure), the free TRAM flap procedure, or the deep inferior epigastric perforator (DIEP procedure).

These techniques utilize a wide range of tissue flaps of various sizes and compositions. For example, with regards to the tissue flaps produced by the TRAM technique, the tissue flap remains attached to the muscle and its blood supply. A modification of the TRAM tissue flap, known as the free TRAM flap, uses a much smaller piece of abdominal muscle; blood is supplied through microsurgical dissection and transplant of blood vessels. In contrast, the DIEP flap procedure takes no muscle at all, relying instead on precise microsurgery to move tiny perforating blood vessels (often a millimeter or less) and then reattach them with sutures finer than human hairs. Regardless of the procedure that utilizes or results in the tissue flap, the present invention can be utilized with respect to such tissue flaps to promote angiogenesis therein and reduce the rate of necrosis in the tissue flaps.

The administration of the viral vector encoding the angiogenic factor and contact with the tissue flap can be accomplished by any suitable method. For example, the aforementioned ex vivo techniques can be utilized. Preferably, the viral vector is administered by direct administration, e.g., injection, of the viral vector into the tissue flap.

The present invention can be used to lower the rate of necrosis within a tissue flap, thereby increasing the survival rate of such flaps. For example, the present invention can lower such rates of necrosis in tissue flaps utilized or formed during surgical procedures, for example created by surgical incision or utilized during primary suturing or skin grafting.

The viral vector of the present invention can be combined with any suitable pharmaceutical carrier. A pharmaceutically acceptable carrier typically will be a substance useful in the administration of the viral vector to an animal, such as a human, for therapeutic treatment.

Specifically, the viral vector can be made into a composition appropriate for contacting cells by combining the viral vector with an appropriate (e.g., pharmaceutically acceptable) carrier such as an adjuvant, vehicle, or diluent. The means of making such a composition, and means of administration, have been described in the art (see, for instance, Remington's Pharmaceutical Sciences, 16th Ed., Mack, ed. (1980)). Where appropriate, the viral vector can be formulated into a preparation in solid, semisolid, liquid, or gaseous form such as tablets, capsules, powders, granules, ointments, solutions, suppositories, injections, inhalants, and aerosols, in the usual ways for their respective routes of administration. Means known in the art can be utilized to prevent release and absorption of the composition until it reaches the target tissue or to ensure timed-release of the composition. A pharmaceutically acceptable form should be employed which does not ineffectuate the viral vector. In pharmaceutical dosage forms, the composition can be used alone or in appropriate association, as well as in combination, with other pharmaceutically active compounds. For example, in applying the method of the present invention for delivery of a nucleic acid sequence encoding VEGF to a tissue flap, such delivery can be employed in conjunction with other means of stimulating angiogenesis, such as, for example, treatment with other angiogenic factors, or use in combination with matrigel (a complex mixture of tumor basement membrane components and growth factors) (see, e.g., Mühlhauser et al., Circ. Res., 77, 1077-86 (1995)).

Accordingly, the pharmaceutical composition can be delivered via various routes and to various sites in an animal body to achieve a particular effect (see, e.g., Rosenfeld et al., Clin. Res., 39(2), 311A (1991a)). One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal administration, as well as topical administration.

The pharmaceutical composition can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and animal subjects, each unit containing a predetermined quantity of the pharmaceutical composition, alone or in combination with other active agents, calculated in an amount sufficient to produce the desired effect. The specifications for the unit dosage forms depend on the particular effect to be achieved and the particular pharmacodynamics associated with the pharmaceutical composition in the particular host.

The “effective amount” of the viral vector to be administered is such as to produce the desired effect, i.e., increased vascularity, in the tissue flap. The desired effect can be monitored using several end-points known to those skilled in the art.

The viral vector can be carried in any suitable volume of pharmaceutically acceptable carrier. The actual dose and administration schedule can vary depending on the nature of the pharmaceutical composition (e.g., whether it contains other active ingredients), as well as interindividual differences in pharmacokinetics, drug disposition, and metabolism. Furthermore, the amount of viral vector to be administered per cell can vary with the nature of the nucleic acid sequence encoding the angiogenic factor, as well as the remainder of the viral vector. As such, the amount of viral vector to be administered per cell desirably is determined empirically, and can be altered due to factors not inherent to the method of the present invention. One skilled in the art can readily make any necessary adjustments in accordance with the exigencies of the particular situation.

With respect to the transfer and expression of a nucleic acid sequence encoding an angiogenic factor according to the present invention, the ordinary skilled artisan is aware that different genetic signals and processing events control levels of nucleic acids and proteins/peptides in a cell, such as, for instance, transcription, mRNA translation, and post-transcriptional processing. Transcription of DNA into RNA requires a functional promoter. The amount of transcription is regulated by the efficiency with which RNA polymerase can recognize, initiate, and terminate transcription at specific signals. These steps, as well as elongation of the nascent mRNA and other steps, are all subject to being affected by various other components also present in the cell, e.g., by other proteins which can be part of the transcription process, by concentrations of ribonucleotides present in the cell, and the like.

Protein expression also is dependent on the level of RNA transcription which is regulated by DNA signals, and the levels of DNA template. Similarly, translation of mRNA requires, at the very least, an AUG initiation codon which is usually located within 10 to 100 nucleotides of the 5′ end of the message. Sequences flanking the AUG initiator codon have been shown to influence its recognition by eukaryotic ribosomes, with conformity to a perfect Kozak consensus sequence resulting in optimal translation (see, e.g., Kozak, J. Molec. Biol., 196, 947-950 (1987)). Also, successful expression of a therapeutic gene in a cell can require post-translational modification of a resultant protein/peptide. Thus, production of a recombinant protein or peptide can be affected by the efficiency with which DNA (or RNA) is transcribed into mRNA, the efficiency with which mRNA is translated into protein, and the ability of the cell to carry out post-translational modification. These are all factors of which the ordinary skilled artisan is aware and is capable of manipulating using standard means to achieve the desired end result.

Along these lines, to optimize production of the angiogenic factor in the tissue flap, the viral vector employed for transfer of the nucleic acid sequence encoding the angiogenic factor further comprises a polyadenylation site following the coding region of the nucleic acid sequence encoding the angiogenic factor. Also, preferably all the proper transcription signals (and translation signals, where appropriate) will be correctly arranged on the viral vector such that the nucleic acid sequence encoding the angiogenic factor will be properly expressed in the cells into which it is introduced. If desired, the viral vector also can incorporate splice sites (i.e., splice acceptor and splice donor sites) to facilitate mRNA production. Moreover, if the nucleic acid sequence encodes an angiogenic factor that is a processed or secreted protein or, for instance, functions in an intracellular organelle, such as a mitochondrion or the endoplasmic reticulum, preferably the viral vector further comprises the appropriate sequences for processing, secretion, intracellular localization, and the like.

With respect to promoters, coding sequences, and other genetic elements located on the viral vector, such elements are as previously described and can be present as part of a cassette, either independently or coupled. A “cassette” is a particular base sequence that possesses functions which facilitate subcloning and recovery of nucleic acid sequences (e.g., one or more restriction sites) or expression (e.g., polyadenylation or splice sites) of particular nucleic acid sequences.

The present inventive method preferably can be employed to a nucleic acid sequence encoding an angiogenic factor that can act locally to stimulate angiogenesis in the setting of tissue ischemia. Viral vector transfer of a nucleic acid sequence encoding an angiogenic factor can be employed to provide a high concentration of the angiogenic factor in a regional fashion for a sustained period, thus inducing angiogenesis in the local milieu, yet minimizing the risk of chronic overinduction of angiogenesis in the target tissue flap, and promiscuous induction of angiogenesis in sensitive nondiseased organs, such as the retina or synovium, or in occult tumors.

### EXAMPLE

This example further illustrates the present invention but should not be construed to limit the present invention in any way. Although this example is recited using particular embodiments, for example using a particular type of viral vector and particular type of angiogenic factor, the skilled artisan will appreciate that the present inventive method can be applied to a wide range of viral vectors and angiogenic factors, using a wide variety of techniques, as described above.

Adenoviral vectors encoding the cDNA for VEGF121 (AdVEGF121) and without VEGF (null vectors) were constructed using standard techniques known in the art. Sprague-Dawley rats (300 g) were divided into three groups (n=10; per group): a control group, null group, and VEGF group. All three groups underwent transverse rectus abdominus myocutaneous (TRAM) flap elevation. The null group was treated with a genetically unmodified adenoviral vector (109 plaque-forming units) by injection into the subcutaneous plane of the inferiorly based TRAM flap two weeks prior to TRAM flap elevation. The VEGF group was treated by injection of AdVEGF121 (109 plaque-forming units) into the subcutaneous plane of the inferiorly based TRAM flap two weeks prior to TRAM flap elevation. The control group received no viral vector prior to TRAM flap elevation.

The TRAM flaps were elevated and inset over silastic barriers. Flap survival was assessed on postoperative day seven by computerized area analysis (statistical analysis by ANOVA), microangiography, and haematoxylin & eosin (H & E) histology.

The majority of observed skin necrosis was contralateral to the deep inferior epigastric pedicle in all three groups. Lead oxide microangiograms showed a large increase in new vessel growth (50-100 μm diameter) in the skin paddle within VEGF treated flaps as compared to the skin paddle in the treatment flaps of the null and control groups. Percentages of surviving flap area in the three groups were determined. The VEGF group showed a significantly greater (p<0.05) percentage of surviving flap area. Specifically, the control group exhibited a 39% surviving flap area, and the null group exhibited a 36% surviving flap area, as compared to the 73% surviving flap area for the VEGF group. The H & E histology also showed increased microvascular density in the VEGF treated flaps.

These results confirm that transfer of a nucleic acid sequence encoding an angiogenic factor via a viral vector to a tissue flap, and expression therein, can be employed to attain a therapeutic effect, namely the increased vascularity of the tissue flap. In particular, the results validate that an adenoviral vector carrying the VEGF cDNA is capable of inducing the growth of new blood vessels within tissue flaps produced during TRAM surgery. This indicates that viral vectors encoding angiogenic factors can fulfill a useful role in the treatment of tissue flaps produced by or used in surgical procedures.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of increasing vascularity in a tissue flap, the method comprising contacting a tissue flap with an adenoviral vector, the adenoviral vector comprising a nucleic acid sequence encoding an angiogenic factor, whereby the nucleic acid sequence encoding the angiogenic factor is expressed in the tissue flap and vascularity in the tissue flap is increased.

3. The method of claim 1, wherein said adenoviral vector is replication-deficient.

4. The method of claim 1, wherein said angiogenic factor is a vascular endothelial growth factor (VEGF).

5. The method of claim 4, wherein the vascular endothelial growth factor is VEGF₁₂₁.

6. The method of claim 1, wherein the adenoviral vector is injected into the tissue flap.

7. The method of claim 1, wherein the rate of necrosis in the tissue flap is decreased by contacting the tissue flap with the adenoviral vector.

8. The method of claim 1, wherein the adenoviral vector is within a pharmaceutically acceptable carrier and the tissue flap is contacted with the pharmaceutically acceptable carrier containing the adenoviral vector.

9. The method of claim 1, wherein the tissue flap is a completely dissociated tissue flap.

10. The method of claim 9, wherein said tissue flap is contacted with adenoviral vector prior to re-association of the tissue flap with an animal host.

11. The method of claim 1, wherein the tissue flap is substantially cut away from surrounding tissue, but is connected to, an animal host.

12. The method of claim 11, wherein the tissue flap is contact with the adenoviral vector prior to re-association of the tissue flap with the surrounding tissue.

13. The method of claim 1, wherein said angiogenic factor is acidic fibroblast growth factor, basic fibroblast growth factor, alpha tumor necrosis factor, beta tumor necrosis factor, platelet-derived growth factor, or angiogenin.
